(19) United States
(12) Patent Application Publication
(10) Pub. No.: US 2017/0252373 A1
(43) Pub. Date: Sep. 7, 2017

(54) COMBINATION THERAPY FOR THE TREATMENT OF HAIR LOSS

(71) Applicant: Nikolai Tankovich, San Diego, CA (US)

(72) Inventor: Nikolai Tankovich, San Diego, CA (US)

(21) Appl. No.: 15/444,829

(22) Filed: Feb. 28, 2017

Related U.S. Application Data
(60) Provisional application No. 62/302,125, filed on Mar. 1, 2016.

(51) Int. Cl. A61K 35/28; A61K 8/49; A61K 8/02; A61K 31/506; A61B 17/54; A61K 9/00; A61Q 7/00; A61N 1/40; A61N 5/06; A61N 7/00; A61K 8/98; A61K 9/70

(52) U.S. Cl. CPC A61K 35/28; A61K 8/981; A61K 8/4953; A61K 8/0216; A61K 31/506; A61K 9/703; A61K 9/0021; A61K 9/0014; A61Q 7/00; A61N 1/40; A61N 5/0617; A61N 7/00; A61B 17/54; A61K 2800/884; A61K 2800/592; A61N 2005/067

(57) ABSTRACT

The invention provides a combination therapy for the treatment of hair loss, the therapy comprising the administration of stem cell factors and vasodilators. The stem cell factors may be obtained from mesenchymal stem cells grown under low oxygen conditions. Kits and compositions for the treatment of hair loss are also disclosed.

### CROSS-REFERENCE TO RELATED APPLICATIONS

This application claims priority to Provisional Application Ser. No. 62/302,125, filed Mar. 1, 2016, the entire contents of which are incorporated by reference for all purposes.

### BACKGROUND

2. Field of the invention

The invention is in the field of treatments for hair loss.

3. Related Art

Hair loss in humans manifests as a result of various causes and to varying degrees. Hair loss may be caused by hormones, age, metabolic syndrome, environmental factors, and genetics. Many causes of male-pattern hair loss remain unknown. Male-pattern hair loss (MPHL), also known as androgenic alopecia and male pattern baldness, is hair loss that occurs due to an underlying susceptibility of hair follicles to shrinkage due to the influence of androgenic hormones. Classic male-pattern hair loss begins above the temples and vertex, or calvaria, of the scalp. As it progresses, a rim of hair at the sides and rear of the head remains. This has been referred to as a ‘Hippocratic wreath’, and rarely progresses to complete baldness.^{[5]} The Hamilton-Norwood scale has been developed to grade androgenic alopecia in males. Male-pattern hair loss is the most common cause of hair loss and will affect up to 70% of men and 40% of women at some point in their lifetimes.^{[1]} Men typically present with progressive hair loss at the temples and vertex balding, whereas women typically present with diffuse hair loss over the top of their scalps.^{[2][3][4]}

Female androgenic alopecia is known colloquially as “female pattern baldness,” although its characteristics can also occur in males. It more often causes diffuse thinning without hairline recession; similar to its male counterpart, female androgenic alopecia rarely leads to total hair loss.^{[6]} The Ludwig scale grades severity of androgenic alopecia in females. Female androgenic alopecia has become a growing problem that, according to the American Academy of Dermatology, affects around 30 million women in the United States. Although hair loss in females normally occurs after the age of 50 or even later when it does not follow events like pregnancy, chronic illness, crash diets, and stress among others, it is now occurring at earlier ages with reported cases in women as young as 15 or 16.^{[7]}

Hair loss can be slowed or reversed in its early stages with medication. Medications approved by the United States' Food and Drug Administration (FDA) to treat male-pattern hair loss include minoxidil and finasteride.^{[8]} Finasteride is a medication of the 5α-reductase inhibitors (5-ARIs) class.^{[9]} By inhibiting type II 5-ARI, finasteride prevents the conversion of testosterone to dihydrotestosterone in various tissues including the scalp. Increased hair on the scalp can be seen within three months of starting finasteride treatment and longer-term studies have demonstrated increased hair on the scalp at 24 and 48 months with continued use.^{[10]} Treatment with finasteride more effectively treats male-pattern hair loss at the vertex than male-pattern hair loss at the front of the head and temples.^{[10]} Dutasteride is a medication in the same class as finasteride but inhibits both type I and type II 5-alpha reductase.^{[10]} Dutasteride is approved for the treatment of male-pattern hair loss in Korea, but not in the United States.^{[10]} However, it is commonly used off-label to treat male-pattern hair loss.^{[10]}

Minoxidil is a growth stimulant that stimulates already-damaged hair follicles to produce normal hair.^{[11]} Minoxidil does not, however, provide any protection to the follicles from further DHT damage, and when a follicle eventually becomes completely destroyed by DHT, minoxidil will no longer be able to have any more regrowth effects on that follicle. Other treatment options include tretinoin combined with minoxidil, ketoconazole shampoo, spironolactone,^{[12]} alfatradiol, and topilutamide (fluridil).^{[13]}

More advanced cases of hair loss may be resistant or unresponsive to medical therapy and require hair transplantation. Naturally occurring units of one to four hairs, called follicular units, are excised and moved to areas of hair restoration.^{[12]} These follicular units are surgically implanted in the scalp in close proximity and in large numbers. The grafts are obtained from either follicular unit transplantation (FUT) or follicular unit extraction (FUE). In the former, a strip of skin with follicular units is extracted and dissected into individual follicular unit grafts. The surgeon then implants the grafts into small incisions, called recipient sites.^{[14][15]}

Many people use unproven treatments including vitamins, minerals, or other dietary supplements, however there is little evidence that these treatments have any benefit^{[16]} and dietary supplements are not typically recommended.^{[17]}

In view of the above, there exists a need for a hair loss treatment that provides lasting results and treats advanced hair loss across the entire region of the scalp and without side effects or the need for surgical intervention.

### SUMMARY OF THE INVENTION

The invention addresses the shortcomings in the art of hair loss treatment by providing a method for treating hair loss comprising administering to a subject in need thereof at least one stem cell factor and at least one vasodilator.

It is therefore an object of the invention to provide a method for treating hair loss in a subject comprising administering to the subject at least one stem cell factor and at least one vasodilator.

A further object of the invention is to provide a kit for treating hair loss in a subject comprising at least one stem cell factor and at least one vasodilator.

A further object of the invention is to provide a composition for treating hair loss in a subject comprising at least one stem cell factor and at least one vasodilator.

A further object of the invention is to provide a microneedle patch for treating hair loss in a subject comprising at least one stem cell factor and at least one vasodilator.

In an aspect of the invention, the vasodilator is minoxidil.

In an aspect of the invention, the stem cell factor is a mesenchymal stem cell factor.

In an aspect of the invention, the mesenchymal stem cell factor is obtained from a mesenchymal stem cell grown under low oxygen conditions.

In an aspect of the invention, the vasodilator is minoxidil.

### DEFINITIONS

The terms “isolated” and “purified” are as used interchangeably herein to refer to the removal of a substance from its natural environment such that it is free or substantially free of other substances, such as substances with which it is normally associated with in nature. An isolated substance can be about 100%, 99%, 98%, 97%, 96%, 95%, 90%, 85%, 80%, 75%, 70%, 65% or 60% free of other substances, as well as any intervening value.

The phrases “low oxygen conditions,” “low oxygen,” “reduced oxygen tension,” and “hypoxia” as used herein refer to any oxygen concentration that is less than atmospheric oxygen. Low oxygen conditions include, but are not limited to, less than about 20%, 19%, 18%, 17%, 16%, 15%, 14%, 13%, 12%, 11%, 10%, 9%, 8%, 7%, 6%, 5%, 4%, 3%, 2%, or 1% oxygen. Low oxygen conditions include, but are not limited to, up to 20%, 19%, 18%, 17%, 16%, 15%, 14%, 13%, 12%, 11%, 10%, 9%, 8%, 7%, 6%, 5%, 4%, 3%, 2%, or 1% oxygen. Low oxygen conditions include, but are not limited to 20% to 19% oxygen, 20% to 18% oxygen, 20% to 17% oxygen, 20% to 16% oxygen, 20% to 15% oxygen, 20% to 14% oxygen, 20% to 13% oxygen, 20% to 12% oxygen, 20% to 11% oxygen, 20% to 10% oxygen, 20% to 9% oxygen, 20% to 8% oxygen, 20% to 7% oxygen, 20% to 6% oxygen, 20% to 5% oxygen, 20% to 4% oxygen, 20% to 3% oxygen, 20% to 2% oxygen, or 20% to 1% oxygen.

As used herein, the phrase “mesenchymal stem cell factor” or “MSCF,” refers to a stem cell factor that is produced by a mesenchymal stem cell.

The term “normoxic” as used herein refers to atmospheric oxygen levels. Normoxic oxygen concentration can be 20% oxygen or above.

As used herein, the phrase “stem cell factor,” or “SCF,” refers to any substance or substances produced through the metabolic activity of a stem cell, including mesenchymal stem cells, endodermal stem cells and ectodermal stem cells. SCF include, but are not limited to, cytokines, chemokines, peptides, proteins, amino acids, polynucleotides (i.e. RNA or DNA), and combinations thereof. CSF can be secreted proteins and/or intracellular proteins.

The term “subject” as used herein typically refers to a human. In certain embodiments, a subject is a non-human mammal. Exemplary non-human mammals include laboratory, domestic, pet, sport, and stock animals, e.g., mice, rats, guinea pigs, cats, dogs, horses, and cows. Typically, the subject is eligible for treatment, e.g., displays one or more indicia of condition being treated.

As used herein, the term “treatment” (and grammatical variations thereof such as “treat” or “treating”) refers to clinical intervention comprising the method(s) of the present invention in an attempt to prevent or alter the course of the disorder being treated. Treating the targeted disorder using the methods disclosed herein may result in one or more of alleviation or amelioration of one or more symptoms of the targeted condition, diminishment of or delay in the appearance of or worsening of any direct or indirect pathological consequences of the targeted condition, decrease of the rate of disease progression of the targeted condition, preventing the progression or manifestation of the targeted condition, and amelioration or palliation of the targeted condition.

### DETAILED DESCRIPTION

The invention provides a combination therapy for treating hair loss. More particularly, the invention provides a method for treating hair loss through the administration of stem cell factors and vasodilators.

In one non-limiting embodiment, the invention comprises a method for treating hair loss in a subject comprising administering to the subject at least one stem cell factor and at least one vasodilator. The subject may have hair loss or be at risk of developing hair loss. The subject may be a subject that has not experienced hair loss, but desires to prevent the onset of hair loss. The subject can have varying degrees of hair loss. The subject may have baldness, thinning of the hair, or a thinning or receding hairline. The subject may be a male. The subject may be a male with male pattern baldness, including without limitation a male subject with hair loss in the Hamilton-Norwood scale ranging from stages Ito VII. The subject may be a female. The subject may be a female that is experiencing the progression of female pattern baldness (androgenic alopecia). The subject may be a female with androgenic alopecia ranging from stages Ito III on the Ludwig scale. Ludwig stage I begins with thinning on the top of the head. In Ludwig stage II the scalp starts to show. With Ludwig stage III, all of the hair at the crown of the head may be lost. The subject may have undergone a hair transplantation procedure.

The invention provides a method for treating hair loss. The phrase “treating hair loss,” and its grammatical equivalents, refers to the application of the methods disclosed herein to achieve a desired effect on the hair of a subject. Such desired effects include, but are not limited to, preventing the onset of hair loss, arresting hair loss, slowing the progression of hair loss, and regrowing hair. The method may be used to maintain hair growth in a subject after the subject has regrown hair following hair loss. The method may be used to maintain hair growth in a subject that has used the method of the invention to regrow hair.

In an aspect of the invention, the method treats hair loss on a target site in a subject. As used herein, the phrase “target site” refers to any site on the body of a subject where the treatment of hair loss is desired. Target sites for use with the method of the invention include, but are not limited to, the scalp, including, but not limited to, the crown, vertex, temples, hairline and/or middle of the head.

In at least one embodiment, the invention provides a method for treating hair loss in a subject comprising administering to the subject at least one SCF and at least one vasodilator. SCF can be SCF obtained from ectodermal stem cells, endodermal stem cells, mesenchymal stem cells (e.g. MSCF), or a combination thereof. The SCF can be MSCF, stem cell factors derived from neural stem cells, or a combination thereof.

MCSF for use with the invention may be obtained (e.g. collected) from any mesenchymal stem cell capable of providing a MCSF having a therapeutic effect in the treatment of hair loss. MCSF can obtained from mesenchymal stem cells having varying degrees of potency, including multipotent mesenchymal stem cells and precursor cells of mesenchymal lineage. MCSF can be obtained from a population of mesenchymal stem cells having the same differentiation potential, or a mixed population of mesenchymal stem cells having varying degrees of differentiation potential. MCSF can be obtained from a population of multipotent mesenchymal stem cells, progenitor cells of mesenchymal lineage, or a combination thereof. MCSF can be obtained from a population of isolated multipotent mesenchymal stem cells, a population of isolated progenitor cells of mesenchymal lineage, or a combination thereof. Such isolated populations of cells can be, for example, cells obtained from the expansion of a clonal cell, or cells purified from a mixed population of cells, such as by FACS.

MSCF can be obtained from mesenchymal stem cells which are grown under in vitro conditions, a population of mesenchymal stem cells that are obtained from the tissue of a donor (e.g. a primary cell culture or tissue explant), or a combination thereof. Mesenchymal stem cells for producing MSCF can be exposed to normoxic conditions, low oxygen conditions, or a combination thereof. Mesenchymal stem cells for producing MCSF can be cultured for one or more passages under low oxygen conditions, under normoxic conditions, or a combination of low oxygen conditions and normoxic conditions. Suitable processes, reagents and equipment for culturing mesenchymal stem cells under low oxygen conditions are disclosed in the following references, the disclosures of which are incorporated herein by reference for all purposes: U.S. Pat. No. 6,759,242; U.S. Pat. No. 6,846,641; U.S. Pat. No. 6,610,540; J. Cereb. Blood Flow Metab. 2008 Sep. 28(9):1530-42; Stem Cells. 2008 May 26(5):1325-36; Exp Neurol. 2008 April 210(2):656-70; Mol. Cell. Neurosci. (2007), doi: 10.1016/j.mcn.2007.04.003; Experimental Neurology 170, 317-325 (2001); and Neurosignals 2006-07, 15:259-265. Although these references disclose particular procedures and reagents, any low oxygen culture condition capable of expanding mesenchymal stem cells may be used. Mesenchymal stem cells for producing MCSF can be exposed to low oxygen conditions, normoxic conditions, or a combination of low oxygen conditions without expanding the mesenchymal stem cells. Such exposure can occur, for example, by incubating the mesenchymal stem cells for a period of time that is less than an amount of time required for the cells to divide, such as in preconditioning. Without limiting the possible embodiments of the invention, low oxygen conditions as disclosed herein can be 5% oxygen.

Mesenchymal stem cells for providing MCSF can be obtained from any source of mesenchymal stem cells capable of providing MCSF that produce a therapeutic effect in the treatment of hair loss when administered according to the methods disclosed herein. Mesenchymal stem cells for providing MCSF for use with the invention include, but are not limited to, mesenchymal stem cells obtained from prenatal sources, postnatal sources, and combinations thereof. Tissues for deriving a suitable source of mesenchymal stem cells for producing MSCF include, but are not limited to, bone marrow, dermis, periosteum, synovium, peripheral blood, skin, hair root (e.g. dermal papilla), muscle, uterine endometrium, adipose, placenta, menstrual discharge, chorionic villus, amniotic fluid and umbilical cord blood. Mesenchymal stem cells for producing MCSF may be derived from these sources individually, or the sources may be combined (before or after enrichment) to produce a mixed population of mesenchymal stem cells from different tissue sources. Mesenchymal stem cells for producing MSCF can be obtained from adult tissues, fetal tissues, differentiated from embryonic or induced pluripotent stem cells, or a combination thereof.

MCSF can be obtained from mesenchymal stem cells that have been expanded in vitro, mesenchymal stem cells obtained from a tissue explant, or a combination thereof. MCSF can be obtained from mesenchymal stem cells purified from a cultured population of mesenchymal stem cells, or purified from a tissue explant.

MCSF can be obtained from the mesenchymal stem cells disclosed in the following references, the disclosures of which are incorporated by reference herein in their entirety for all purposes: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal, N., et al., J. Cell Biochem. (1997) 64(2): 295 312: Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Mesenchymal stem cells for providing MCSF may be obtained from donor sources that are allogeneic, syngeneic, or xenogeneic with respect to the subject that is to be treated with the MSCF. In some embodiments, the MSCF are obtained from a human donor source that is allogeneic with respect to the subject to be treated. The MSCF from allogeneic donors of the mesenchymal stem cells can be HLA matched with the subject that is to be treated.

MSCF can be obtained from homogenates, including homogenates of mesenchymal stem cells grown in culture, homogenates of mesenchymal stem cells purified from a tissue source, or homogenates of tissues containing mesenchymal stem cells. MSCF can be obtained from mesenchymal stem cell conditioned medium. MSCF can be obtained from the foregoing homogenates, mesenchymal stem cell conditioned medium, or a combination thereof. MSCF can comprise conditioned medium. MCSF can be conditioned medium that has been used to grow mesenchymal stem cells under low oxygen, low serum conditions. MCSF can be conditioned medium that has been used to grow mesenchymal stem cells under chronic low oxygen. As used herein, the term “chronic low oxygen” refers to culturing stem cells exclusively under low oxygen conditions, beginning with passage 0. The low oxygen conditions can be 1-10% oxygen. The low oxygen conditions can be 5% oxygen. In at least one embodiment, the MSCF are obtained from mesenchymal stem cell conditioned medium. The mesenchymal stem cell conditioned medium can be medium that was used to grow mesenchymal stem cells under low oxygen conditions, normoxic oxygen conditions, or a combination thereof. The mesenchymal stem cell conditioned medium can be medium that was used to grow mesenchymal stem cells under low oxygen, low serum conditions. As used herein, the term “low serum,” or “low serum conditions,” refers to the culture of cells in a culture medium that comprises less than about 5% serum. Low serum conditions include growing cells in a culture medium having a concentration of about 0.1% and 0.2% serum. The serum can be obtained from sources including, but not limited to, human, bovine, goat, pig, horse, rabbit, rat, and combinations thereof. MCSF can be conditioned medium as disclosed herein, wherein the conditioned medium is preserved by vaporization, such as, according to the methods disclosed in U.S. Pat. No. 9,469,835, the entire contents of which are incorporated herein by reference in their entirety.

MSCF can include at least one of: BLC, Eotaxin-1, Eotaxin-2, G-CSF, GM-CSF, I-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Axl, BTC, CCL28, CTACK, CXCL16, ENA-78 (CXCLS), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP. The methods and compositions disclosed herein can be practiced with one or more of the MSCF disclosed in this paragraph. The methods and compositions disclosed herein can be practiced with all the MSCF disclosed in this paragraph. The invention can be practiced with any combination of the MSCF disclosed in this paragraph, wherein one or more of the foregoing MSCF are specifically excluded. MSCF can comprise functional fragments, analogs or derivatives of the MSCF disclosed herein.

In one non-limiting embodiment of the invention, the invention is practiced with MSCF comprising ICAM-1, IL-6, IL-8, IL-15, IL-16, OPN, TIMP-1, TIMP-2, TNF RI, PF4, MCF R, BMP-5, EGF R, bFGF, FGF-4, FGF-7, HGF, IGFBP-1, IGFBP-2, IGFBP-3, IGFBP-4, IGFBP-6, OPG, Insulin, IGF-I, SCF, MCSF, VEGF, Axl, CXCL16, ENA-78 (CXCL5), GRO, IL-29, MCP-1, MDC, MIF, and GCP-2.

It will be appreciated that the invention can be practiced with one or more of the following cellular factors which are obtained from a source (e.g. cell) other than a stem cell or mesenchymal stem cell: BLC, Eotaxin-1, Eotaxin-2, G-CSF, GM-CSF, 1-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Axl, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP. Sources other than stem cells and mesenchymal stem cells for the foregoing cellular factors include non-mesenchymal stem cells grown in culture (e.g. clonal cell lines) or recombinant sources

The stem cell factors disclosed herein can be obtained from recombinant sources by transfecting cells with one or more polynucleotides that encode the stem cell factors that are desired. Stem cells that naturally express a desired stem cell factor may be transfected to achieve greater expression of the desired stem cell factor, such as transfection to achieve constitutive expression of the desired stem cell factor.

The stem cell factors can be lyophilized stem cell factors. Stem cell factors may be lyophilized by any means suitable for producing stem cell factors that produce a therapeutic effect when administered to a subject according to the methods disclosed herein. Stem cell factors may be vaporized according to the methods disclosed in U.S. Patent Application Publication Numbers 2008/0229609 and 2010/0120014, the entire contents of which are incorporated by reference herein in their entirety for all purposes.

In at least one embodiment, the method of the invention comprises administering at least one stem cell factor and at least one vasodilator. The SCF and vasodilator can be present in a ratio of about 1:10 and 1:100, as well as any intervening ratio, such as, for example, 1:50 or 1:25. Vasodilators for use with the invention may be synthetic or natural compounds. Natural vasodilators for use with the invention include, but are not limited to, nitrates, flavonoids, and L-arginine. The vasodilator can be minoxidil (e.g. 2,4-pyrimidinediamine, 6-(1-piperidinyl)-, 3-oxide). The minoxidil can be a 5%-10% solution. Suitable vasodilators for use with the invention include, but are not limited to, abnormal cannabidiol, adenosine, amrinone, amyl nitrite, arteriolar vasodilator, bamethan, benziodarone, betahistine, buflomedil, butalamine, carbocromen, carpronium chloride, cilostazol, cinaciguat, cinepazet, clonidine, cloridarol, cromakalim, diazoxide, dihydroergocristine, dilazep, doxazosin, efloxate, endothelium-derived hyperpolarizing factor, etafenone, etofylline nicotinate, flosequinan, gapicomine, heptaminol, hexobendine, histamine, hydralazine, imolamine, inositol nicotinate, isosorbide dinitrate/hydralazine, isoxsuprine, itramin tosilate, linsidomine, milrinone, minoxidil, molsidomine, naftidrofuryl, nicorandil, nicotinyl alcohol, nitrovasodilator, oxyfedrine, papaverine, prazosin, pentifylline, pentoxifylline, phentolamine, pimobendan, prenylamine, quazinone, regadenoson, riociguat, sodium nitroprus side, tanakan, terazosin, tezosentan, tolazoline, trapidil, vinburnine, vinpocetine, visnadine, xanthinol, xantinol nicotinate, yohimbine, or a combination thereof.

In at least one embodiment, the method of the invention comprises administering to a subject in need of treatment for hair loss at least one stem cell factor and at least one vasodilator. The stem cell factor and the vasodilator may be administered sequentially, or simultaneously. When administered simultaneously, the stem cell factor and vasodilator may optionally be administered as a single composition. The stem cell factor and vasodilator may be administered one or more times. The stem cell factor and vasodilator may be administered to a target site where the treatment of hair loss is desired. The stem cell factor and vasodilator may be administered to the target site topically and/or by injection. The stem cell factor may be administered topically, and the vasodilator may be suitable for oral administration, such as a natural vasodilator, and may be administered orally, such as for example as a nutritional supplement.

The stem cell factors and vasodilators disclosed herein can be formulated with a pharmaceutically acceptable carrier. The stem cell factors and vasodilators can be formulated with a carrier for administration orally, parenterally, sublingually, transdermally, rectally, transmucosally, topically, via inhalation, via buccal administration, intrapleurally, intravenously, intraarterially, intraperitoneally, subcutaneously, intramuscularly, intranasally, intrathecally, intravaginally, retrobulbarly, intraarticularly, or a combination thereof. In at least one non-limiting embodiment, the stem cell factor and vasodilator are formulated for topical administration to a target site. In at least one non-limiting embodiment, the stem cell factor and vasodilator are formulated for injection at a target site. The stem cell factors and vasodilators can be formulated individually with at least one pharmaceutically acceptable carrier. The stem cell factors and vasodilators may be combined with one another and at least one pharmaceutically acceptable carrier. Suitable pharmaceutically acceptable carriers for use with the invention include those disclosed in Remington's Pharmaceutical Sciences, 19th Edition, Mack Publishing Co., Easton, Pa. 1995, the entire contents of which are incorporated herein by reference in their entirety for all purposes. The SCF and vasodilators can be combined with one or more artificial pharmaceutically acceptable carriers. In one non-limiting embodiment, lyophilized (e.g. vaporized) SCF are suspended in one or more pharmaceutically acceptable carriers. The SCF and/or vasodilator may be formulated in any format suitable for topical application to a target site. The SCF and/or vasodilator may be formulated as a foam, cream, paste, lotion, gel, emulsion, liquid, spray, or powder. The SCF and vasodilator can be formulated in a shampoo or hair conditioner.

In some embodiments, the invention provides a kit for the treatment of hair loss. The kit can comprise at least one SCF and at least one vasodilator. The kit can comprise at least one isolated SCF and at least one vasodilator. The SCF and vasodilator can be present in a ratio of about 1:10 and 1:100, as well as any intervening ratio, such as, for example, 1:50 or 1:25. The kit can comprise at least one SCF that is lyophilized or vaporized as disclosed herein, and at least one vasodilator, wherein the vasodilator is optionally in the form of a powder, or is lypopholized or vaporized, and wherein the kit further comprises at least one pharmaceutically acceptable carrier for formulating the at least one SCF and at least one vasodilator for topical administration or injection. The kit can contain one or more containers of SCF and one or more containers of the vasodilator. The SCF for the kit can be MSCF derived from mesenchymal stem cell conditioned medium. The MSCF can be derived from mesenchymal stem cell conditioned medium that has been used to grow mesenchymal stem cells under low oxygen, low serum culture conditions. The SCF and vasodilator can be combined to form a single composition. The composition can be formulated for topical administration, such as in the form of, for example, a foam, cream, paste, lotion, gel, sol, emulsion, liquid, spray, or powder. In at least one embodiment, the kit comprises at least one device for preparing a target site for the topical administration of the SCF and vasodilator. The kit may comprise one or more of a laser, a device that emits radiofrequency and/or ultrasonic energy, a mechanical oscillator, a dermabrasion device, dermabrasion particles, needle, and a needle roll.

In one non-limiting embodiment, the invention provides a kit comprising: at least one vasodilator; at least one MSCF, wherein the MSCF is derived from mesenchymal stem cell conditioned medium that has been used to grow mesenchymal stem cells under low oxygen, low serum culture conditions; wherein the vasodilator and SCF are in the form of a powder or are lyophilized or vaporized; and a pharmaceutically acceptable carrier suitable for administering the vasodilator and MSCF topically or by injection.

In one non-limiting embodiment, the invention provides a kit for treating hair loss comprising at least one vasodilator and at least one MSCF obtained from mesenchymal stem cells grown under low oxygen conditions, and wherein the at least one MSCF comprises one or more of: BLC, Eotaxin-1, Eotaxin-2, G-CSF, GM-CSF, 1-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Axl, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP.

In at least one embodiment, the invention provides a microneedle patch comprising at least one SCF and at least one vasodilator. The SCF may be MSCF that are obtained from mesenchymal stem cells grown under low oxygen, low serum conditions. The SCF and vasodilator can be present in a ratio of about 1:10 and 1:100, as well as any intervening ratio, such as, for example, 1:50 or 1:25. The microneedle patch can be a patch disclosed in US Patent Application Publication No. 2016/0287668, the entire contents of which are incorporated herein by reference for all purposes. The patch can contain a combination of at least one vasodilator and at least one MCSF as disclosed herein.

### EXAMPLE

A Caucasian male approximately 62 was treated with a combination of 5% minoxidil and mesenchymal stem cell conditioned medium obtained from bone marrow mesenchymal stem cells grown constantly under 5% oxygen conditions beginning with passage 0. The conditioned medium was preserved by vaporization until placed in solution immediately before application. The combination of minoxidil and conditioned medium was applied topically for three months to the scalp, including temples and crown. The subject experience a reduction in hair loss, thickening of hair, increased hair pigmentation, conversion of vellus hairs to regular anagen hairs, reduction in the appearance of scalp skin showing through the hair, and overall increase in hair density.

### REFERENCES

1. Yim, Elizabeth; Nole, Katherine L. Baquerizo; Tosti, Antonella (December 2014). “5α-Reductase inhibitors in androgenetic alopecia.”. Current Opinion in Endocrinology, Diabetes, and Obesity (Review) 21 (6): 493-8.

2. McElwee, K. J.; Shapiro, J. S. (2012). “Promising therapies for treating and/or preventing androgenic alopecia”. Skin therapy letter 17 (6): 1-4.

3. Proctor, P. H. (1999). “Hair-raising. The latest news on male-pattern baldness”. Advance for nurse practitioners 7 (4): 39-42,83.

4. Leavitt, M. (2008). “Understanding and Management of Female Pattern Alopecia”. Facial Plastic Surgery 24 (4): 414-427.

5. Hippocratic wreath (Baldness)”. Britannica Online. Dec. 15, 2012. Retrieved Dec. 15, 2012.

6. “Female pattern baldness”. MedlinePlus. Dec. 15, 2012. Retrieved Dec. 15, 2012.

7. Women and Hair Loss: The Causes”. Archived from the original on 30 Jun. 2010. Retrieved 2010-06-29.

8. “Propecia (Finasteride) Drug Information: User Reviews, Side Effects, Drug Interactions and Dosage”. RxList. Mar. 13, 2010. Retrieved Nov. 28, 2010.

9. Supenya, Varothai (July 2014). “Bergfeld”. Wilma F. 15 (3): 217-30.

10. Yim, Elizabeth; Nole, Katherine L. Baquerizo; Tosti, Antonella (December 2014). “5α-Reductase inhibitors in androgenetic alopecia.”. Current Opinion in Endocrinology, Diabetes, and Obesity (Review) 21 (6): 493-8.

11. “Minoxidil solution”. DermNet N Z. Dec. 29, 2013. Retrieved Jan. 29, 2014.

12. Rogers, N. E.; Avram, M. R. (2008). “Medical treatments for male and female pattern hair loss”. Journal of the American Academy of Dermatology 59 (4): 547-566; quiz 566-8.

13. Varothai, Supenya; Bergfeld, Wilma F. (2014). “Androgenetic Alopecia: An Evidence-Based Treatment Update”. American Journal of Clinical Dermatology 15 (3): 217-230.

14. Caroli, S.; Pathomvanich, D.; Amonpattana, K.; Kumar, A. (2011). “Current status of hair restoration surgery”. International surgery 96 (4): 345-351.

15. “The Latest Innovations in Hair Transplantation”. Facial Plastic Surgery 27 (4): 366-377.

16. Levy, L L; Emer, J J (29 Aug. 2013). “Female pattern alopecia: current perspectives.”. International journal of women's health 5: 541-56.

17. Rogers, N E; Avram, M R (October 2008). “Medical treatments for male and female pattern hair loss.”. Journal of the American Academy of Dermatology 59 (4): 547-66; quiz 567-8.

1. A method for treating hair loss in a subject comprising administering to a target site on the subject at least one stem cell factor and at least one vasodilator, wherein administering the at least one stem cell factor and at least one vasodilator treats hair loss in the subject.

2. The method of claim 1, wherein the at least one stem cell factor comprises mesenchymal stem cell factors.

3. The method of claim 2, wherein the mesenchymal stem cell factors are bone marrow mesenchymal stem cell factors.

4. The method of claim 2 or 3, wherein the mesenchymal stem cell factors are ischemic tolerant mesenchymal stem cell factors.

5. The method of any one of claims 1-4, wherein the mesenchymal stem cell factors are human mesenchymal stem cell factors.

6. The method of any one of claims 1-5, wherein the at least one stem cell factor is obtained from stem cells that have been cultured under low serum conditions.

7. The method of any one of claims 1-6, wherein the at least one stem cell factor and the at least one vasodilator are administered in a ratio of between about 1:10 and 1:100.

8. The method of any one of claims 1-7, wherein the at least one vasodilator is selected from the group consisting of doxazosin, prazosin, terazosin, clonidine, hydralazine, and minoxidil.

9. The method of any one of claims 1-8, wherein the stem cell factors and minoxidil are administered simultaneously.

10. The method of any one of claims 1-8, wherein the at least one stem cell factor and the at least one vasodilator are administered sequentially.

11. The method of any one of claims 1-9, wherein the at least one stem cell factor and the at least one vasodilator are combined.

12. The method of any one of claims 1-11, wherein the at least one stem cell factor and the at least one vasodilator are combined with at least one pharmaceutically acceptable carrier.

13. The method of any one of claims 1-12, wherein the at least one stem cell factor is vaporized.

14. The method of any one of claims 1-13, wherein the at least one stem cell factor and the at least one vasodilator are applied topically.

15. The method of any one of claims 1-14, wherein administering produces permanent neovascularization in the site.

16. The method of any one of claims 1-15, wherein the at least one stem cell factor and at least one vasodilator are in the form of a liquid, gel, powder, foam, cream, bandage, microneedle patch, or paste.

17. The method of any one of claims 1-16, wherein the target site is treated with at least one of radiofrequency, laser energy, ultrasonic energy, a mechanical oscillator, dermabrasion device, dermabrasion particles, needle, and a needle roll before administering one or more of the at least one stem cell factor and the at least one vasodilator.

18. The method of any one of claims 1-17, wherein administering prevents hair loss or regrows hair on the subject.

19. A composition for treating hair loss in a subject, the composition comprising at least one stem cell factor and at least one vasodilator.

20. The composition of claim 19, wherein the at least one stem cell factor comprises mesenchymal stem cell factors.

21. The composition of claim 20, wherein the mesenchymal stem cell factors are bone marrow mesenchymal stem cell factors.

22. The composition of claim 20 or 21, wherein the mesenchymal stem cell factors are ischemic tolerant mesenchymal stem cell factors.

23. The composition of any one of claims 19-22, wherein the at least one stem cell factor is a human stem cell factor.

24. The composition of any one of claims 19-23, wherein the at least one stem cell factor is obtained from stem cells that have been cultured under low serum conditions.

25. The composition of any one of claims 19-24, wherein the at least one stem cell factor and at least one vasodilator are in a ratio of between about 1:10 and 1:100.

26. The composition of any one of claims 19-25, wherein the at least one vasodilator is selected from the group consisting of doxazosin, prazosin, terazosin, clonidine, hydralazine, and minoxidil.

27. The composition of any one of claims 19-26, wherein the at least one stem cell factor and the at least one vasodilator are combined.

28. The composition of any one of claims 19-27, wherein the at least one stem cell factor and the at least one vasodilator are combined with at least one pharmaceutically acceptable carrier.

29. The composition of any one of claims 19-28, wherein the at least one stem cell factor is vaporized.

30. The composition of any one of claims 19-29, wherein the composition is contained in two-phase dispensing vessel.

31. The composition of any one of claims 19-29, wherein the composition is contained in a patch.

32. The composition of claim 31, wherein the patch is a microneedle patch.

33. The composition of any one of claims 19-32, wherein the at least one stem cell factor and the at least one vasodilator are formulated for topical administration.

34. The composition of any one of claims 19-33, wherein the stem cell factors and the at least one vasodilator are in the form of a liquid, gel, foam, cream, or paste.

35. A kit for treating hair loss in a subject, the kit comprising the composition of any one of claims 19-34 and a device for administering at least one of laser energy, a radiofrequency or ultrasonic energy to a target site on a subject.
